(19) United States
(12) Patent Application Publication
(10) Pub. No.: US 2020/0046458 A1
(43) Pub. Date: Feb. 13, 2020

(54) ENDODONTIC PIN

(71) Applicant: Stephan Lampl, Kriessern (CH)

(72) Inventor: Stephan Lampl, Kriessern (CH)

(21) Appl. No.: 16/494,368

(22) PCT Filed: Mar. 8, 2018
(86) PCT No.: PCT/EP2018/055724

§ 371 Date: Sep. 16, 2019

(30) Foreign Application Priority Data
Mar. 14, 2017 (AT) A 98/2017

(51) Int. Cl. A61C 5/35; A61K 6/00

(52) U.S. Cl. CPC A61C 5/35; A61K 6/0044; A61K 6/0091

(57) ABSTRACT

An endodontic pin (1) having a base (2), wherein the base (2) has a root portion (3) for fastening the base (2) in a natural tooth root (4) and a crown portion (5) for fastening a dental crown facing (6) to the endodontic pin (1) and/or for arrangement in a natural tooth crown (32). The base (2) is formed of a composite material (7) which includes at least one organic binder (8), the composite material (7) including glass particles (9) as a filler.

### TECHNICAL FIELD

The present invention relates to an endodontic pin having a base pin, wherein the base pin has a root portion for fastening the base pin in a natural tooth root and a crown portion for fastening a tooth crown facing to the endodontic pin and/or for arranging in a natural tooth crown, and the base pin is composed of a composite material having at least one organic binder.

### BACKGROUND

In endodontics, a branch of dentistry, a remnant of a natural tooth or at least of a natural tooth root is used to fasten a root pin or endodontic pin with its root portion in the tooth root. The crown portion of the endodontic pin is used to stabilize and/or otherwise supplement a remnant of a natural tooth crown and/or to fasten a tooth crown facing to the crown portion of the endodontic pin.

Endodontic pins made of various materials are known in the prior art. For example, there are many endodontic pins made of metal, e.g. titanium and the like. A problem here is, on the one hand, that the mechanical properties of these metals deviate markedly from the natural tooth substance. On the other hand, there are also problems regarding the biocompatibility of metals, since deposition of metal ions in the tooth and/or in the surrounding soft tissue has been a critical issue.

The prior art also comprises endodontic pins of the type in question in which glass fibers or carbon fibers are embedded in the organic binder. However, experience has shown that signs of fatigue and especially a separation of fibers and binder may occur in these fiber-reinforced synthetics.

### SUMMARY

The problem addressed by the invention is therefore to improve an endodontic pin of the abovementioned type in such a way its mechanical properties are as similar as possible to the natural tooth substance and in such a way that it has the longest possible useful life.

To this end, the invention provides that the composite material has glass particles as a filler.

An underlying concept of the invention is therefore to produce the endodontic pin using a composite material, or composite, which on the one hand has an organic binder and on the other hand is filled with glass particles as filler. It has been found that in this way endodontic pins can be provided which have mechanical properties very similar to the natural tooth substance and which also have a sufficiently long useful life. Preferred variants of endodontic pins according to the invention have, as the finished end product, a flexural strength in the range of 150 MPa (megapascal) to 250 MPa, preferably of 180 MPa to 220 MPa, particularly preferably 200 MPa. The compressive strength of such endodontic pins is expediently in the range of 450 MPa to 550 MPa, preferably in the range of 500 to 550 MPa, particularly preferably 550 MPa. The flexural modulus of such endodontic pins is expediently in the range of 12 GPa (gigapascal) to 25 GPa, preferably of 17 GPa to 25 GPa, particularly preferably 20 GPa. The surface hardness of preferred endodontic pins is expediently in the range of 85 HV (Vickers hardness) to 120 HV, preferably of 90 HV to 100 HV, particularly preferably 95 HV. All of these parameters can be determined according to EN ISO 4049, edition: 2013 Mar. 1.

The grain sizes of the glass particles of the composite material are preferably in the range of 0.01 μm (micrometer) to 50 μm, particularly preferably in the range of 0.01 μm to 3 μm. The glass particles can be rounded, but they can also have a particle shape deviating from the latter. For the tightest possible packing, the composite material preferably has glass particles of different grain sizes. The composite material can have glass particles of a single glass type, but also of different glass types. These are preferably what are known as macrofilled composite materials. The volume fraction of the glass particles in the finished end product of the endodontic pin or base pin expediently measures at least 78% by volume, preferably at least 83% by volume, particularly preferably at least 90% by volume. The glass particles are preferably ones that have or consist of barium glass and/or strontium glass. The glass particles expediently have a silanized surface. Barium glass and strontium glass are particularly materials that are inherently radiopaque, such that the endodontic pin can be imaged by X-ray even after application in the tooth of the patient. If the glass particles themselves are not radiopaque, aluminum oxide or the like can also be added as further filler to the composite material in order to create suitable radiopacity and therefore visibility in an X-ray image. As further filler, zinc oxide (ZnO) can be added to the composite material of the endodontic pin in order to achieve an antibacterial action. The volume fraction of the zinc oxide in the endodontic pin, i.e. in the end product, is expediently ca. 2-5%. Further fillers of the composite material can be pigments, i.e. coloring agents, and/or catalysts for supporting the polymerization of the organic binder, and/or other additives.

In preferred embodiment variants of the invention, the organic binder includes or consists of at least one methacrylate. The organic binder preferably has methacrylate at least as the main constituent.

The product designated REF 2061, available from Indigodental GmbH & Co. KG in Pinneberg, Germany, can be used for example as the composite material for the production of the base pin or endodontic pin. This product contains triethylene glycol dimethacrylate, urethane dimethacrylate, bis-GMA, and ethoxylated bisphenol A dimethacrylate. The product REF 2050, for example, from the same company can be used as the organic binder.

An endodontic pin is a pin that can be used in endodontics. An endodontic pin according to the invention can also be referred to as a root pin. The endodontic pin according to the invention can be formed exclusively of the base pin. However, endodontic pins according to the invention can also have further constituent parts, e.g. the attachment casing mentioned further below. The base pin has at any rate a root portion. This is the part of the base pin that is anchored in a natural tooth root in order to fasten the base pin. For this purpose, a corresponding canal is generally created in the natural tooth root, e.g. by drilling, into which canal the root portion of the base pin is inserted. The base pin additionally has a crown portion. This is the part that is to be arranged in the region of a natural tooth crown of a natural tooth, or in most cases of a remaining part of the latter. However, the crown portion can also serve for fastening a tooth crown facing, particularly if there remains absolutely nothing or just remnants of the natural tooth crown. Endodontic pins according to the invention can therefore be used, on the one hand, to fasten a tooth crown facing, i.e. an artificial tooth crown or an artificial part of a tooth crown, on a natural tooth root. However, endodontic pins according to the invention can also be used to stabilize a remaining part of the natural tooth crown and serve as an anchor for artificial parts of the tooth crown that are optionally to be added. In this sense, the tooth crown facing can thus replace only parts of a natural tooth crown or else the entire natural tooth crown. In order to fasten the root portion of the base pin in the tooth root, the nerve canal often present originally in the natural tooth root, and in most cases drilled a little further, is used as a canal for fastening the root portion in this canal.

Endodontic pins or root pins according to the invention are preferably prefabricated mass-produced items that can be manufactured cost-effectively and that the dentist can fit at a single visit into the remaining tooth root or into the remaining natural tooth. Since the composite material of the endodontic pin according to the invention is very similar to the natural tooth substance in terms of its mechanical properties, the drills, grinders and the like that are familiar to a dentist can be used for any shortening or shaping work that may be needed before fitting.

The root portion of the base pin is preferably bonded or cemented with a fastening material, or in other words a cement, into the previously prepared and cleaned canal of the natural tooth root. The fastening material used for this purpose preferably has a composition similar to that of the composite material of the base pin. The fastening material is thus expediently also a composite material with an organic binder and glass particles as filler. Thus, the organic binder of the fastening material can preferably again include or be composed of at least one methacrylate. However, since the fastening material in the uncured state should be liquid or at least pasty, provision is expediently made that the degree of filling, i.e. the volume fraction of the fillers, in particular of the glass particles, is less than in the composite material of the endodontic pin. The proportion of filler, in particular the proportion of glass particles, in the fastening material is expediently between 45% by volume and 65% by volume.

By virtue of the proportion of organic binder of the fastening material, the latter can be polymerized by the action of light and thus cured. In order to support and promote the best possible permanent connection and rapid curing of the fastening material, provision is made, in particularly preferred variants of endodontic pins according to the invention, that the base pin has a light admission surface in the crown portion and, in order that light introduced via the light admission surface is conveyed onward into the root portion, is designed to be light-transmitting at least in part, preferably all over. Therefore, in these embodiments, the base pin itself is used as a kind of light guide, such that light introduced into the base pin via the light admission surface is guided via the base pin itself to the fastening material, in order to force the curing of the fastening material.

In order to provide suitably good light transmission, the base pin in preferred embodiments has a relatively low opacity at least in part, preferably all over, or to put it the other way a relatively high degree of translucence. The opacity of the base pin at least in part, preferably all over, is preferably in a range of between 15% and 45%, preferably in a range of 25% to 30%, particularly preferably ca. 27%. This opacity, and all the opacities mentioned below, can be determined according to DIN 6174. The light admission surface is expediently located at the end of the crown portion of the base pin remote from the root portion.

To ensure that the light introduced via the light admission surface is guided particularly effectively to the places where it is needed for curing the fastening material or cement material, the base pin in preferred variants has a lens body. This lens body is expediently located in the crown portion of the base pin. The lens body is preferably configured as a thickening of the base pin. This means that the lens body therefore preferably has a diameter greater than the root portion and also greater than the rest of the crown portion of the base pin. The lens body is distinguished by its refractive action. It diverts the light, introduced via the light admission surface, in such a way that said light is conveyed to the places where it is needed for curing the fastening material. These are in particular the outer surfaces of the root portion of the base pin, and also any shoulder portions of the endodontic pin that may be present, which shoulder portions are explained in more detail below and can likewise bear on the remaining natural tooth root.

In preferred embodiments, the root portion of the base pin is designed as a cone tapering in a direction away from the crown portion. It is preferably a root portion that is rotationally symmetrical with respect to a longitudinal axis of the base pin. The cone is thus expediently frustoconical or conical. The outer surface of the root portion of the base pin can have a certain roughness. However, there are preferably no outwardly visible structures such as threads or the like in the outer surface of the root portion of the base pin. In preferred embodiments, it could thus be said that the outer surface of the root portion is smooth.

As has been stated, endodontic pins according to the invention can be formed exclusively of the base pin. However, in other embodiments of the invention, provision can also be made that the endodontic pin has, in addition to the base pin, an attachment casing which envelops, preferably exclusively, the crown portion of the base pin, preferably in a circumferentially closed manner. Provision is preferably made here that the attachment casing has a higher opacity than the base pin. The higher opacity of the attachment casing in relation to the base pin serves to ensure that the complete or partial crown that is ultimately created is as similar as possible, also in terms of color, to the natural appearance of a tooth crown. The opacity of the attachment casing is expediently in the range of 50% to 80%, preferably 65% to 75%, particularly preferably ca. 70%.

Apart from the pigments, i.e. coloring agents, needed for its color, the attachment casing can be made of the same composite material as the base pin or can at least have such a material. To color the composite material used for producing the attachment casing, organic and/or inorganic pigments known per se can be used, e.g. also whiteners such as TiO2. The pigments can be adapted such that the overall appearance of the attachment casing is as close as possible to that of the natural dentine in the mouth of the patient. Expressed using the Lab color space, the attachment casing preferably has an L value of 59 to 69 and/or an a value of −0.35 to −3.2 and/or a b value of −0.4 to −6.95. Like the determination of the opacity, the determination of the L value, the a value and the b value can be carried out according to the provisions of DIN 6174. In preferred embodiments of such endodontic pins, provision is made that the attachment casing, which could also be designated as a restorative attachment casing, forms a shoulder portion of the endodontic pin, which shoulder portion protrudes relative to the root portion of the base pin. This shoulder portion is expediently formed in a closed manner around the circumference of the whole endodontic pin. With this shoulder portion, the endodontic pin can bear on the remaining natural tooth root.

In preferred embodiment of the invention, provision is made that the base pin is of an inherently monolithic configuration. This preferably also applies to the optionally present attachment casing. Monolithic here means produced from one casting or in one piece. There are therefore expediently no interfaces in the base pin and if appropriate also in the attachment casing. The base pin is therefore preferably formed from one piece, and the composite material is preferably uniform throughout the inside of the base pin. The same preferably applies also to the attachment casing.

In particularly preferred embodiments of endodontic pins according to the invention, provision is made that the glass particles are fused together. The glass particles of the composite material of the endodontic pin are thus still discernible as such in corresponding microscopic images but are interconnected by melting and fusing together of their surfaces. This fusing together of the glass particles can be achieved by a laser treatment of the base pin and, if appropriate, also of the attachment casing, as is explained in more detail below. The base pin and/or also the optionally present attachment casing can thus be laser treated, as can be seen from the fusing together of the glass particles. The fusing together results in a still stronger bond than the one already provided by the connection of the glass particles by means of the cured organic binder. In preferred embodiments, the glass particles are fused together in the whole base pin. If an attachment casing is present, this also applies in preferred embodiments to the attachment casing. Thus, the glass particles are also expediently fused together in the whole of the attachment casing if present.

To be able to create in the remaining natural tooth root a canal matching the root portion of the base pin, drills are expediently made available which are adapted in shape to the shape of the root portion of the base pin. Here, adapted means that the drills are able to drill, into the remaining natural tooth root, a hole or a canal that provides the space needed to fasten therein the root portion of the base pin and the necessary fastening material. The shape of the drill thus corresponds substantially to the shape of the root portion of the base pin or can be slightly larger in order to create space also for the fastening material. In this context, the invention also relates to a kit having at least one endodontic pin according to the invention, preferably several endodontic pins according to the invention, and at least one drill, wherein the shape of the drill is adapted to the shape of the root portion of the base pin of the endodontic pin. Such a kit can of course also comprise several endodontic pins and also, if necessary, several drills matching them. In addition, the kit can also comprise the appropriate fastening material. The drills can have a longitudinal stop and/or a preferably visible marking, such that the hole is always drilled to the optimal length or depth in the tooth root.

### BRIEF DESCRIPTION OF THE DRAWINGS

Further features and details of preferred embodiments of endodontic pins according to the invention and of methods for their production are explained in more detail below in the description of the figures, in which:

FIGS. 1 and 2 show two side views of a base pin according to the invention, from two directions rotated 90° relative to each other;

FIGS. 3 to 7 show various illustrative embodiments of endodontic pins according to the invention, with a base pin according to FIGS. 1 and 2 and with various attachment casings;

FIGS. 8 to 13 show examples of the use of the endodontic pins from FIGS. 1 to 7 in teeth;

FIG. 14 shows a schematic enlarged view of the composite material of the endodontic pin according to the invention;

FIGS. 15 and 16 show views of production methods, and

FIG. 17 shows a schematic view of a drill with a shape adapted to the base pin.

### DETAILED DESCRIPTION

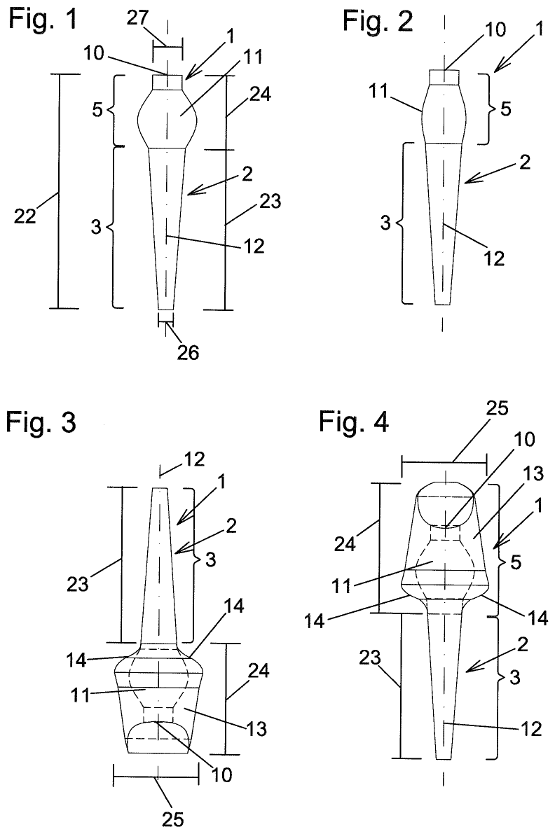

The example of an endodontic pin 1 according to the invention shown in FIGS. 1 and 2 is formed exclusively of the base pin 2. The base pin 2 has a root portion 3 and a crown portion 5. The root portion 3 is intended to be fastened in a corresponding canal 33 in a remaining tooth root 4 of a natural tooth. The crown portion 5 of the base pin 2 is for arrangement in a natural tooth crown or, if the latter is no longer present or if only part of it is present, serves to fasten a tooth crown facing 6 on the endodontic pin 1 or on the base pin 2. The root portion 3 is designed as a cone tapering in a direction away from the crown portion 5 and is designed rotationally symmetrically with respect to the longitudinal axis 12 of the base pin 2. The crown portion 5 of the base pin 2 can likewise be designed rotationally symmetrically with respect to the longitudinal axis 12 of the base pin 2. It is not so designed in the illustrative embodiment shown, as can be seen by comparing FIGS. 1 and 2. FIG. 2 shows the side view from a direction rotated 90° relative to FIG. 1. In these illustrative embodiments, the crown portion 5 ends in the light admission surface 10 at its side remote from the root portion 3. In this illustrative embodiment, the thickening 11, which forms a lens body, is also located in the crown portion 5. In the preferred embodiments as shown here, the entire base pin 2 is so transparent or translucent that it transmits light. As a result of this, light introduced via the light admission surface 10 is conveyed inside the base pin 2 as far as the root portion 3. The thickening 11, functioning as a lens, refracts the light into the root portion 3 and thus intensifies the light-transmitting effect. The lens formed by the thickening 11 can also be configured such that it conveys the light not only into the root portion 3 but also into the shoulder portion 14 of the embodiment variants of endodontic pins 1 shown in FIGS. 3 to 7. This light-transmitting design of the base pin 2 permits particularly good and rapid curing of fastening material 28 with which the root portion 3 of the base pin 2 is fastened in the tooth root 4. The whole base pin 2 is of a monolithic design, i.e. from one casting. It is composed, according to the invention, of a composite material 7 which has at least an organic binder 8 and glass particles 9 as filler. Furthermore, the composite material 7 can additionally have the other fillers already mentioned in the introduction. The glass particles 9 are expediently fused together and are thus firmly and permanently interconnected, on the one hand by the organic binder 8 arranged between them, and on the other hand by the fusing together. A structure of the base pin 2 is obtained that has the particularly favorable mechanical properties already set out in the introduction.

FIGS. 3 to 7 show illustrative embodiments of endodontic pins 1 according to the invention in which the endodontic pins 1 each have a base pin 2 and an attachment casing 13. In all of these illustrative embodiments, the respective attachment casings 13 circumferentially envelop the crown portion 5 of the respective base pin 2. In the preferred embodiments, the attachment casing 13 in each case has a higher opacity than the base pin 2. Each of these casing attachments 13 has a shoulder portion 14 which is here likewise circumferentially closed and protrudes in relation to the root portion 3 of the base pin 2. All of the illustrative embodiments according to FIGS. 3 to 7 have identically configured base pins 2 onto which the respective attachment casing 13 has been applied in a manner to be described below. In preferred embodiments, like the ones shown here, the attachment casing 13, apart from pigments for increasing the opacity, is made of the same composite material 7 as the base pin 2. The pigments are chosen such that the color of the attachment casing 13 is as close as possible to the natural coloring of dentine. The connection between the attachment casing 13 and the base pin 2 is provided not only by the form-fit engagement but also by curing of the organic binder 8 of the attachment casing 13. In addition, in preferred embodiments, the glass particles 9 of the base pin 2 and of the attachment casing 13 are also fused together in the transition region between base pin 2 and attachment casing 13.

The region of the base pin 2 arranged inside the attachment casing 13 in the embodiments of FIGS. 3 to 7, that is to say substantially the crown portion 5 of the base pin 2, is shown only by broken lines in FIGS. 3 to 7, since it is not visible from the outside in the perspective shown. An exception to this is in each case the light admission surface 10 of the base pin 2. This is intended to be kept free of the attachment casing 13 in a manner accessible from the outside, such that light is introduced into the light admission surface 10 and can be conveyed, via the thickening 11 functioning as lens, into the root portion 3 and preferably also into the shoulder portions 14. Like the base pin 2, the attachment casing 13 is also preferably monolithic, i.e. formed from one casting. In the attachment casing 13 too, the glass particles 9 of the composite material 7 are preferably also not interconnected only by the cured organic binder 8 but also by being fused together. If present, the attachment casing 13 can be used as an enlargement and/or as an adaptation of the shape of the crown portion 5 to the particular type of tooth to be treated. Thus, with the aid of the attachment casing 13, the contact surface of the endodontic pin 1 with the tooth crown facing 6 can be enlarged in relation to the crown portion 5 of the base pin 2. In such embodiment variants of the invention, the tooth crown facing 6 is then fastened on the crown portion 5 of the base pin 2 with interpositioning of the attachment casing 13, i.e. fastened indirectly as it were. Furthermore, the attachment casing 13, by having a suitable coloring, can also be used to adapt the color of those parts of the respective endodontic pin 1 that protrude from the tooth root 4.

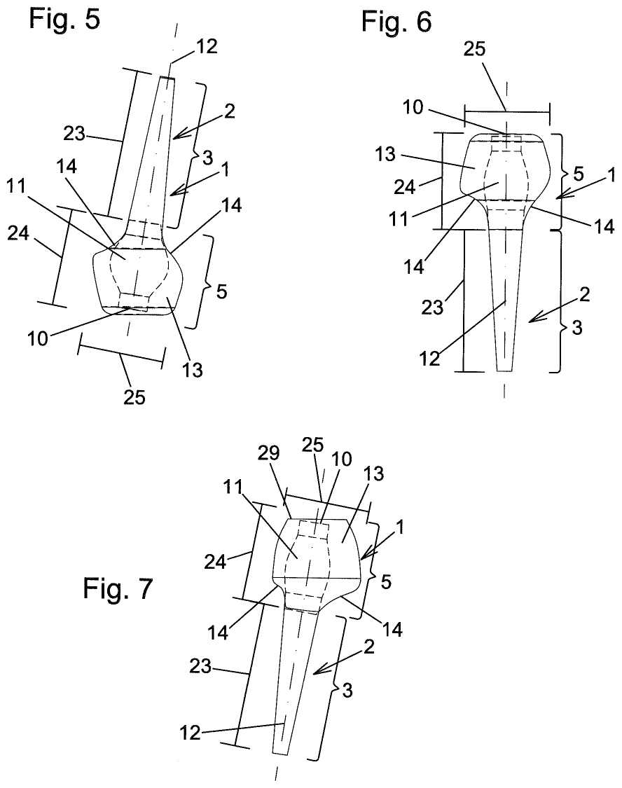

FIG. 3 shows and illustrative embodiment of an endodontic pin 1 which is provided specially for an upper front tooth. FIG. 4 shows an embodiment variant of an endodontic pin 1 for a lower front tooth. FIGS. 5 and 6 show embodiment variants of endodontic pins 1 for upper (FIG. 5) and lower (FIG. 6) premolars. FIG. 7 shows an embodiment variant for a lower molar. For molars, it has proven expedient if the longitudinal axis 12 of the root portion 3 of the base pin 2 is oriented at an angle of expediently 79° with respect to an upper cover surface 29 of the attachment casing 13.

The length 23 of the tooth portion 3 of the base pin 2 is preferably in a range of 11 mm to 13 mm, preferably 12 mm. The length 24 of the crown portion 5 of the base pin 2 expediently measures between 3 mm and 5 mm, preferably 4 mm. The overall length 22 of the base pin 2 accordingly results from the sum of these two values. At its lower end directed away from the crown portion 5, the root portion 3 expediently has a width 26 of between 1.0 mm and 1.5 mm. This end is preferably a blunt end, i.e. not a pointed end. The width 27 at the end of the crown portion 5 of the base pin 2 remote from the root portion 3 preferably measures between 1.8 mm and 2.2 mm, preferably 2 mm. In variants with an attachment casing 13, as shown by way of example in FIGS. 3 to 7, the length 23 of the root portion 3 can also be slightly shorter in relation to the base pin 2, in a range of 9 mm to 11 mm, preferably being 10 mm. In preferred embodiments, the width 30 of the attachment casing 13 can vary, for example, between 4 mm and 9 mm. The length 31 of the attachment casing 13 can likewise vary, for example between 4 mm and 10 mm. The stated lengths are measured parallel to the longitudinal axis 12, while the stated widths are measured at right angles to the latter.

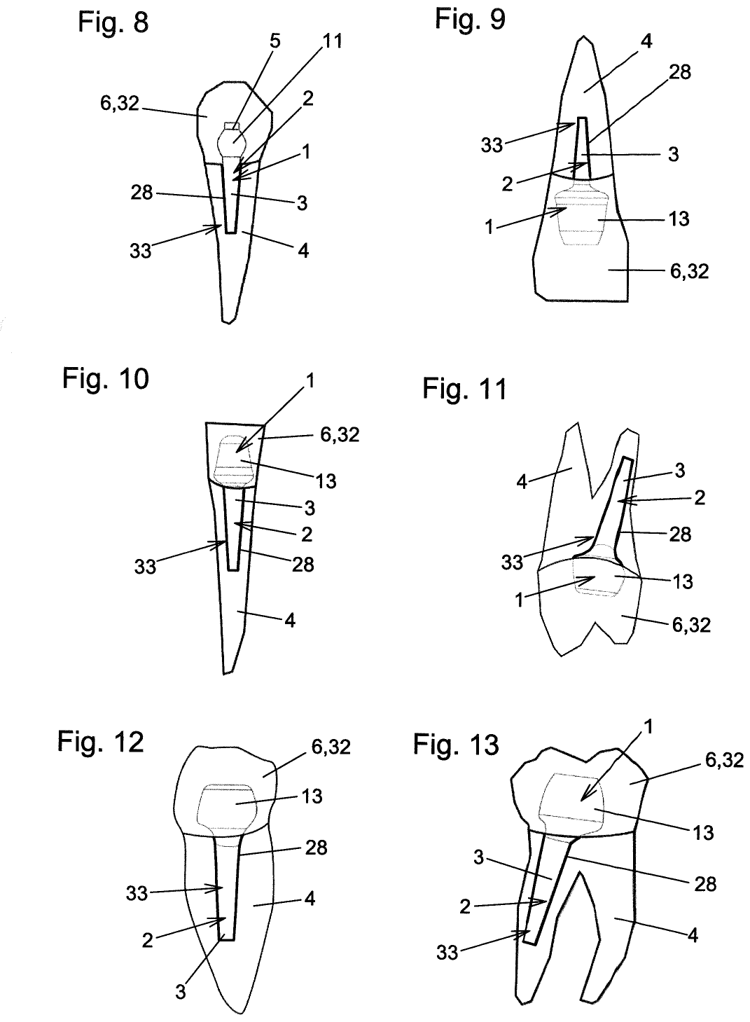

FIGS. 8 to 13 show schematic views of various teeth into which the various illustrative embodiments of endodontic pins 1 according to the invention, as shown in FIGS. 1 to 7 have been applied. Of the teeth, at least a sufficiently large part of the natural tooth root 4 still has to be present in order that the root portion 3 of the endodontic pin 1 can be fastened in a preferably drilled canal 33 of the natural tooth root 4. The natural tooth crown 32 originally present may be wholly or partially missing. The missing parts of the tooth crown 32 are replaced in FIGS. 8 to 13 by corresponding tooth crown facings 6, i.e. artificially manufactured parts. Depending on the embodiment according to the invention, the tooth crown facings 6 are fastened to the endodontic pin 1 either directly on the crown portion 5 or indirectly, with interpositioning of an attachment casing 13 on the crown portion 5. The teeth are each shown in transparent form in FIGS. 8 to 13, such that the endodontic pin 1 is seen in the interior of the natural tooth. In the schematic views in these figures, it is also not specified which parts of the tooth crown 32 still is formed of natural substance and which parts have been replaced by a tooth crown facing 6. As has been stated, this can vary, depending on the individual circumstances, between total replacement of the natural substance and replacement of relatively small parts of the natural sub stance.

FIG. 8 shows the use of the endodontic pin 1 according to FIGS. 1 and 2, which is composed exclusively of the base pin 2. FIG. 9 shows the use of the endodontic pin 1 according to FIG. 3, and FIG. 10 shows the use of the endodontic pin 1 according to FIG. 4. FIGS. 9 and 10 each show front teeth. FIGS. 11 and 12 each show the arrangement of the endodontic pins from FIGS. 5 and 6 in a premolar. FIG. 13 shows a molar in which the endodontic pin 1 according to FIG. 7 has been applied.

The natural tooth root 4 into which a canal 33 has been drilled is shown in each case. The respective endodontic pin 1 is cemented or bonded in the canal 33 by the fastening material 28. Before the tooth crown facing 6 is applied or the tooth crown 32 completed, the fastening material 28 can be cured by light being introduced into the respective light admission surface 10, since the suitably light-transmitting base pin 2 of the respective endodontic pin 1 conveys the light, needed for accelerated curing of the fastening material 28, from the light admission surface 10 into the root portion 3 and if appropriate also into the shoulder portion 14 and thus directly to the fastening material 28. As soon as the respective endodontic pin 1 is suitably fastened in the natural tooth root 4, a tooth crown facing 6 can then be applied to the crown portion 5 of the base pin 2 and, if present, to the attachment casing 13 of the endodontic pin 1. These tooth crown facings 6 are known per se. As is known in the prior art, they can be produced and fastened to the respective crown portion 5 or attachment casing 13 by methods known per se. However, the tooth crown facing 6 can also take the form of veneers, i.e. front tooth facings, or onlays, i.e. artificial bite surfaces. However, if regions of the natural tooth crown 32 are still present, they can be supplemented by methods known per se for building up a tooth crown facing 6 and can be built up again to a complete tooth crown 32, after insertion of the endodontic pin 1 into the tooth root 4. The crown portion 5, along with the attachment casing 13 if present, is then arranged in the natural tooth crown 32 and serves to stabilize the latter and acts as a fastening point for the artificially added parts of the tooth crown 32.

For the sake of completeness, it will be noted that, as is also shown in FIGS. 8 to 13, it is entirely possible that the root portion 3 of the base pin 2 also protrudes a distance into the tooth crown facing 6 or into the tooth crown 32. It may also be a good thing in practice that the attachment casing 13 or the crown portion 5 and in particular also the shoulder portion 14 are recessed a distance into the natural tooth root 4.

FIGS. 11 and 13 show that, in the premolars and molars, in which of course several natural tooth roots 4 may be present, the corresponding root portion 3 of the base pin 2, or of the endodontic pin 1, is generally arranged only in one tooth root 4.

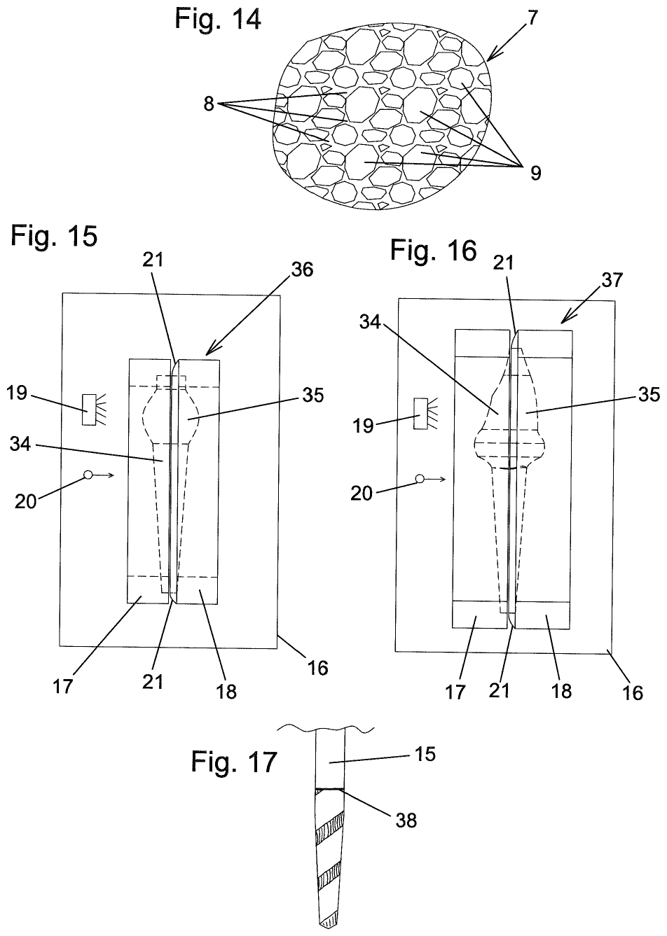

FIG. 14 shows a schematic and greatly enlarged view of the composite material 7 of the base pin 2 and also of the attachment casing 13. It illustrates the very tight packing of the glass particles 9 with the organic binder 8 arranged between them. In preferred embodiments, however, the glass particles 9 are not only held together by the organic binder 8 but are also fused together at their surfaces. The other possible fillers mentioned at the outset, such as zinc oxide, pigment, catalyst and/or additives, are not shown in FIG. 14, since they generally account for only very small volume fractions of the overall composite material 7.

A possible method for producing endodontic pins 1 according to the invention is explained by way of example below. Individual steps of the method, e.g. the polymerization by the use of the LED lamp 19 and/or the laser irradiation for fusing the glass particles 9, can also be combined with method steps other than those explained below.

First of all, the individual constituent parts of the composite material 7, i.e. primarily the organic binder 8 and the preferably silanized glass particles 9 and if appropriate other fillers such as zinc oxide, catalysts and/or additives, are mixed with one another. Alternatively, as has already been explained in the introduction, it is also possible to use commercially available ready-made mixtures of the composite material 7.

The production of a base pin 2 is now explained with reference to FIG. 15. Since the base pin 2 in preferred embodiments is intended to be designed to transmit light, it must have a correspondingly high degree of transparency or translucence. Accordingly, the composite material 7 for the base pin 2 has no or only very small amounts of pigments, i.e. coloring agents. The ready-mixed or commercially available composite material 7 is now first of all heated to a temperature of 80° C. and, in a clean room, is introduced in sufficient quantity into an open mold 36, in the illustrative embodiment shown into the second mold half 18 thereof. The second mold half 18 is preferably made of metal, in particular of steel. The surface delimiting the mold cavity 35 of this mold half 18 can be reflective. The first mold half 37, which is preferably made of glass or of another suitably light-transmitting material, is then pressed onto the second mold half 18, such that the mold 36 composed of the mold halves 17 and 18 is closed. The cavities 34 and 35 in the two mold halves 17 and 18 predefine the shape of the base pin 2 to be produced and have to be filled completely with composite material 7. When the mold halves 17 and 18 are closed, excess material 7 is pressed out between them. Upon closure of the mold halves 17 and 18, the bevels 21 shown in this illustrative embodiment ensure that no flash forms on the base pin 2, which flash would then have to be removed.

Alternatively to this, the composite material 7 could of course also be introduced by injection molding into an already closed mold.

In preferred embodiments of the method, the filled and closed mold is then heated to a temperature of 300° C. and maintained at this temperature for at least half an hour. In this way, a temperature-induced polymerization process takes place for curing the organic binder 8 in the composite material 7. To further promote the polymerization or curing of the organic binder 8, an LED lamp 19 is provided in the illustrative embodiment shown. The light of the LED lamp 19, which preferably lies in a wavelength range of 395 to 480 nanometers, particularly preferably of 450 to 480 nanometers, passes through the light-transmitting first mold half 17 to the composite material 7 in the mold cavities 34 and 35 and further promotes the polymerization or curing of the organic binder 8. The LED lamp 19 can be so large that it covers the whole of the first mold half 17. Of course, it is equally possible also to move the LED lamp 19 along the first mold half 17 in order to expose the whole mold cavity 34, 35 uniformly to LED light.

Following this process of curing or polymerization of the organic binder 8, the composite material 7 in the mold cavities 35 and 35 is then also subjected to laser treatment in order to fuse the glass particles 9 of the composite material together. For this purpose, the composite material 7 in the mold cavities 34 and 35 is subjected to laser light from the laser light source 20 by way of the first mold half 17. The intensity and the duration of irradiation are set such that the laser light passes through the whole of the composite material 7 and, in this way, the glass particles 9 in the composite material 7 are fused together in the whole of the base pin 2 located in the mold halves 17 and 18. For this purpose, the laser light source 20 can be suitably guided across the whole of the first mold half 17 or the whole region of the mold cavities 34 and 35. After completion of the laser treatment, the base pin 2 produced can be removed from the mold halves 17 and 18. As a result of the curing of the organic binder 8 and the subsequent fusion of the glass particles 9, in particular at their surfaces, a monolithic base pin 2 is preferably produced. If the endodontic pin 1 that is to be produced according to the invention is intended to be composed exclusively of the base pin 2, the production process is thereby concluded, although this does not of course rule out secondary treatments, e.g. of the surface.

By contrast, if the endodontic pin 1 according to the invention is intended also to have an attachment casing 13, the finished base pin 2 is introduced into a second mold 37 shown in FIG. 16. This also takes place in a clean room 16 with the mold halves 17 and 18 opened. The composite material 7 provided for the attachment casing 13, which contains the corresponding pigments and can otherwise have the same starting material as in the base pin 2, is first of all heated, as in the production of the base pin, to a temperature of 80° C. and is then introduced into the regions of the mold cavities 34 and 35 of the second mold 37 that remain alongside the base pin 2. Thereafter, the two mold halves 17 and 18 are pressed onto each other such that the mold 37 is closed. Upon closure of the mold 37, the bevels 21 have the same effect as in the mold 36. The subsequent curing of the organic binder 8 and the preferably provided laser treatment for fusion of the glass particles 9 in the attachment casing 13 can then be carried out analogously to the corresponding method steps in the production of the base pin 2. By virtue of the light-transmitting properties of the base pin 2, no shadows occur, such that both the LED light and also the laser light can pass through the whole attachment casing 13 and, therefore, a monolithic attachment casing 13 can also be created. In these method steps, the corresponding connection of the attachment casing 13 to the base pin 2 is also obtained automatically. After completion of these method steps, to be carried out as in the production of the base pin 2, the finished endodontic pin 1 can be removed from the mold halves 17 and 18 of this second mold 37. Here too, some secondary working, e.g. of the surface of the attachment casing 13, is of course also possible, although not absolutely necessary.

FIG. 17 shows a schematic view of a drill 15, the shape of which is adapted, e.g. by a suitably conical configuration, to the shape of the root portion 3 of the respective base pin 2 of the endodontic pin 1. Therefore, by using this drill 15, a canal 33 optimally matching the shape of the root portion 3 is created in the respective tooth root 4. In order to ensure the optimal depth of the canal 33, the drill 15 can have a preferably optically discernible marking 38, e.g. a colored marking, and/or it can also have a corresponding longitudinal stop (not shown here).

In this connection, provision can be made that one or more endodontic pins 1 are sold in a kit together with suitably matching drills 15. This kit can additionally comprise the appropriate fastening material 28.

### KEY TO THE REFERENCE NUMBERS

1 endodontic pin
2 base pin
3 root portion
4 tooth root
5 crown portion
6 tooth crown facing
7 composite material
8 organic binder
9 glass particle
10 light admission surface
11 thickening
12 longitudinal axis
13 attachment casing
14 shoulder portion
15 drill
16 clean room
17 first mold half
18 second mold half
19 LED lamp
20 laser light source
21 bevel
22 overall length
23 length
24 length
25 width
26 width
27 width
28 fastening material
29 upper cover surface
30 width
31 length
32 tooth crown
33 canal
34 mold cavity
35 mold cavity
36 first mold
37 second mold
38 marking

1. An endodontic pin, comprising:
a base pin including a root portion adapted for fastening the base pin in a natural tooth root, and a crown portion adapted for at least one of fastening a tooth crown facing to the endodontic pin or for arranging in a natural tooth crown, and
the base pin is composed of a composite material having at least one organic binder and glass particles as filler.

2. The endodontic pin as claimed in claim 1, wherein the glass particles have or are comprised of at least one of barium glass or strontium glass.

3. The endodontic pin as claimed in claim 1, wherein the organic binder comprises or is at least one methacrylate.

4. The endodontic pin as claimed in claim 1, wherein the base pin further comprises a light admission surface in the crown portion and, in order that light introduced via the light admission surface is conveyed onward into the root portion, and the base pin is at least partially light-transmitting.

5. The endodontic pin as claimed in claim 1, wherein the base pin includes a lens body.

6. The endodontic pin as claimed in claim 1, wherein the root portion of the base pin is formed as a cone tapering in a direction away from the crown portion.

7. The endodontic pin as claimed in claim 1, wherein the endodontic pin has, additionally to the base pin, an attachment casing which envelops the crown portion of the base pin.

8. The endodontic pin as claimed in claim 7, wherein the attachment casing forms a shoulder portion of the endodontic pin that protrudes relative to the root portion of the base pin.

9. The endodontic pin as claimed in claim 1, wherein the base pin is of an inherently monolithic configuration with the glass particles being fused together.

10. A kit comprising at least one endodontic pin as claimed in claim 1 and at least one drill, wherein a shape of the drill is adapted to a shape of the root portion of the base pin of the endodontic pin.

11. The endodontic pin as claimed in claim 1, wherein the glass particles have a silanized surface.

12. The endodontic pin as claimed in claim 2, wherein the glass particles have a silanized surface.

13. The endodontic pin as claimed in claim 3, wherein the composite material further comprises a filler including at least one of zinc oxide, at least one pigment, at least one catalyst, or at least one additive.

14. The endodontic pin as claimed in claim 5, wherein the lens body forms a thickening of the base pin.

15. The endodontic pin as claimed in claim 5, wherein the lens body is in the crown portion of the base pin.

16. The endodontic pin as claimed in claim 1, wherein the root portion is formed as a body that is rotationally symmetrical with respect to a longitudinal axis of the base pin.

17. The endodontic pin as claimed in claim 7, wherein the attachment casing has a higher opacity than the base pin.

18. The endodontic pin as claimed in claim 7, wherein the attachment casing is comprised of a same composite material as the base pin and pigments.

19. The endodontic pin as claimed in claim 7, wherein the base pin and the attachment casing are of an inherently monolithic configuration with the glass particles being fused together.
